(19) United States
(12) Patent Application Publication
(10) Pub. No.: US 2014/0275849 A1
(43) Pub. Date: Sep. 18, 2014

(54) SYSTEM AND METHOD FOR MONITORING AND DIAGNOSING A PATIENT CONDITION BASED ON WIRELESS SENSOR MONITORING DATA

(71) Applicant: Peerbridge Health, Inc., New York, NY (US)

(72) Inventor: ANGELO JOSEPH ACQUISTA, New York, NY (US)

(73) Assignee: Peerbridge Health, Inc., New York, NY (US)

(21) Appl. No.: 14/215,965

(22) Filed: Mar. 17, 2014

Related U.S. Application Data
(60) Provisional application No. 61/787,772, filed on Mar. 15, 2013.

(51) Int. Cl. A61B 5/00; A61B 5/0205

(52) U.S. Cl. CPC A61B 5/0022; A61B 5/0205
USPC 600/301

(57) ABSTRACT

A system including wireless sensors for monitoring a patient's vital signs, such as various hemodynamic parameters of the patient. The system includes a plurality of wireless sensors attachable to or implantable in the subject, each sensor including a sensing component configured to detect a signal corresponding to at least one hemodynamic condition of the subject, and a communication component configured to wirelessly transmit the detected signal to another of the plurality of wireless sensors. The system also includes a master node configured to receive signals from and transmit control commands to at least one of the plurality of wireless sensors. A method of determining a patient's health status by utilizing data gathered from the wireless sensors as well as information of the patient from an electronic medical record storage and management system, is also provided.

### CROSS-REFERENCE TO RELATED APPLICATION

This application claims priority to U.S. provisional application No. 61/787,772, filed on Mar. 15, 2013, the disclosure of which is incorporated herein by reference in its entirety.

### FIELD OF THE INVENTION

The present invention relates to one or more wireless sensors, and a network of wireless sensors for monitoring, in real time (or quasi real time), a patient's vital signs, such as various hemodynamic parameters of the patient. Further, the present invention relates to integration of such wireless sensors with an electronic medical record storage and management system for managing patient healthcare, such as providing clinical decision support, facilitating diagnosis and validating treatment options.

### BACKGROUND

Monitoring various vital signs of a patient has been an important aspect of hospital patient care, especially for patients with diseases at advanced stages, suffering from severe trauma, or in other emergency settings. Additionally, outpatient monitoring of various physiological conditions are being increasingly used for evaluation of patient health conditions as well as early detection and treatment of heart diseases, diabetes, and other diseases. For example, an electrocardiogram (ECG or EKG) can be used to evaluate the heart condition of a patient, where electrodes are placed at certain locations on the chest, arms, and/or legs. These electrodes can be connected to an ECG machine by lead wires, and the electric signals received by the ECG machine can be analyzed and displayed for the physician's information and further interpretation.

Attempts have also been made to develop systems to improve a patient's comfort, freedom and privacy by decreasing the number and volume of devices directly or indirectly attached to the patient. For example, U.S. Pat. No. 7,979,111, the disclosure of which is incorporated in its entirety by reference herein, discloses a wireless electrode arrangement and method for patient monitoring, where a plurality of wireless electrodes suitable for attachment to the surface of the body of a patient are capable of continuously monitoring of a subject wirelessly.

Since the last decade, and especially after the enactment of the American Recovery and Reinvestment Act of 2009, healthcare providers are facing more regulations regarding electronic record management (EMR) and electronic health records (EHR) (or personal health record (PHR)). Meanwhile, medical software providers have been developing a plethora of systems that facilitate electronic data storage and management to enable healthcare providers to be in compliance with such increased regulations. For example, a patient's EHR can provide a longitudinal electronic record of patient health information gathered during one or more encounters in a care delivery setting, which can include information such as patient demographics, medications, vital signs, medical history, laboratory test results, and radiology reports, etc. The EHR can also be used to provide decision support, quality management, and outcomes reporting.

There is a need for a system that integrates the real time monitoring capability of a network of wireless sensors worn by a patient with the data storage and processing capabilities afforded by electronic health records management systems for personalized monitoring and clinical decision support, improving accuracy in diagnosis and validating treatment options proposed by physicians.

### SUMMARY OF THE INVENTION

According to some embodiments of the present invention, a system for monitoring a subject is provided. The system includes a plurality of wireless sensors attachable to or implantable in the subject, each sensor comprising a sensing component configured to detect a signal corresponding to at least one hemodynamic condition of the subject, and a communication component configured to wirelessly transmit the detected signal to another of the plurality of wireless sensors. The system further comprises a control node or master node configured to receive signals from at least one of the plurality of wireless sensors. The master node can include a monitoring unit and a communication component.

In some embodiments of the system, the plurality of wireless sensors are configured to form a mesh network.

In some embodiments of the system, the vital signs include hemodynamic parameters selected from one or more of pulse oximetry, oxygen saturation, oxyhemoglobin saturation, blood glucose level, blood pressure, blood velocity, blood flow rate, respiratory rate, pulse rate, CO₂ level, drug concentration, blood protein concentration, heart rate, heart rhythm, heart rate variability, organic or inorganic substance concentration, cardiac activity, cardiac output, pH levels, pathogens and galvanic skin response.

In some embodiments of the system, at least one of the plurality of wireless sensors of the system includes a sensing component configured to detect a signal corresponding to a first hemodynamic parameter of the subject, and at least another of the plurality of wireless sensors includes a sensing component configured to detect a signal corresponding to a second hemodynamic parameter of the subject, the second hemodynamic parameter being different from the first hemodynamic parameter.

In some embodiments of the system, at least one of the plurality of sensors is implantable. In certain embodiments of the system, at least one of the plurality of sensors is configured to be attachable to the skin of a patient. In certain embodiments, the plurality of sensors comprise at least one surface-attachable sensor and at least one implantable sensor. The at least one surface-attachable sensor can receive signals from the at least one implantable sensor, and relay the signals to a master node.

In some embodiments of the system, the sensing component includes at least one of an electromagnetic detector, a thermal detector, a pressure detector, an ultrasonic detector, an optical detector and a chemical detector, a magnetic detector, a laser detector, and an x-ray detector. In some embodiments of the system, the monitoring device is a portable computing device.

According to some embodiments of the present invention, a method of monitoring a condition for a subject is provided, which includes: (a) detecting signals relevant to one or more vital signs of a subject by one or more wireless sensors attached to the skin or implanted in the body of the subject; (b) wirelessly receiving, by a computing device, the signals from the one or more wireless sensors; (c) making a diagnosis utilizing a processor of the computing device regarding a condition of the subject based on the signals received from the wireless sensors; (d) accessing, by the computing device, at least one medical record of the subject; and (e) determining, by the computing device, a health status of the subject based on the diagnosis made in step (c) and the medical record accessed in step (d). In certain embodiments, the signals are live signals acquired by the one or more wireless sensors in real time. For example, the signals are for the vital signs relevant to one or more prescribed therapies of the subject reflected by the at least one medical record. In certain embodiments, the method further comprises sending an alert to a physician according to the health status, and/or validating a prescription for the subject by a physician based on the health status determined in step (e). In some embodiments, the at least one medical record is stored on one of the plurality of wireless sensors.

### BRIEF DESCRIPTION OF THE DRAWINGS

The various aspects and embodiments disclosed herein will be better understood when read in conjunction with the appended drawings, wherein like reference numerals refer to like components. For the purposes of illustrating aspects of the present application, there are shown in the drawings certain preferred embodiments. It should be understood, however, that the application is not limited to the precise arrangement, structures, features, embodiments, aspects, and devices shown, and the arrangements, structures, features, embodiments, aspects and devices shown may be used singularly or in combination with other arrangements, structures, features, embodiments, aspects and devices. The drawings are not necessarily drawn to scale and are not in any way intended to limit the scope of this invention, but are merely presented to clarify illustrated embodiments of the invention. In these drawings:

FIG. 1 is a schematic block diagram of a network structure including a plurality of wireless sensors and a master node and communication modes therebetween in accordance with one embodiment of the present invention; and

FIG. 2 is an illustrative electrical design of a wireless sensor in accordance with one embodiment of the present invention.

### DETAILED DESCRIPTION

Certain embodiments of the present invention will now be discussed with reference to the aforementioned figures. In one embodiment, the present invention provides a system for managing healthcare for a subject (or a patient). The system includes a plurality of wireless sensors suitable for attachment to the skin of a subject or implantable in the body of the subject. The plurality of wireless sensors preferably form a network. The sensors include a sensing component configured to detect a signal corresponding to at least one physiological condition of the subject, and a communication component configured to wirelessly transmit the detected signal to at least one of the plurality of wireless sensors as well as wirelessly receive a signal transmitted from at least one of the remaining sensors in the network.

As described herein, a wireless sensor includes a sensing component configured to detect a signal corresponding to a physiological condition, such as vital signs including hemodynamic parameters of a patient. Hemodynamics, as known in the art, relates to the study of blood flow. The circulatory system, including the heart, the arteries, the microcirculation, and the vein, functions to transport the blood to deliver O₂, nutrients and chemicals to the cells of the body, and to remove the cellular waste products. The heart is the driver of the circulatory system generating cardiac output (CO) by rhythmically contracting and relaxing. This creates changes in regional pressures, and, combined with a complex valvular system in the heart and the veins, ensures that the blood moves around the circulatory system in one direction. Hemodynamic parameters (or properties), as described herein, include the physiological conditions associated with the blood flow, which includes not only the physical characteristics of the blood flow itself, e.g., blood flow rate, blood flow pressure, and pulse rate, but also those parameters relating to the blood components such as cells, proteins, chemicals, etc.

The vital signs to be monitored as contemplated in the disclosed subject matter can include, but are not limited to, ECG (electrocardiogram), EEG (electroencephalogram), EMG (electromyogram), EOG (electrooculogram), ERG (electroretinogram), temperature, pulse oximetry, oxygen saturation, oxyhemoglobin saturation, blood component concentration (e.g., glucose level, lipid level, cholesterol level, triglyceride level, levels of different salts, concentration of different types of cells, concentration of blood proteins such as thrombin, cancer markers, heart failure markers), renal function test components (e.g., concentration of albumin, urea, and creatinine in the urine), liver function test components, organ functions, blood pressure (such as atrial pressure, ventricular pressure, pulmonary artery pressure, systolic pressure, diastolic pressure, etc.), blood velocity, respiration rate, pulse rate, (end tidal) CO₂ level, blood drug concentration, organic or inorganic substance concentration in the blood (e.g. uric acid, vitamins, heavy metals, carbon monoxide, bacterial toxin), cardiac output, heart rate, heart rhythm, heart rate variability, pH, pathogens, motion, weight, etc. Additionally, the system can be used to monitor migraine, patient's galvanic skin response, and responses to electrical nerve and muscle stimulation, etc. Depending on the types of underlying physiological conditions to be monitored, the sensing component can include, but is not limited to, an electrochemical detector (such as an needle electrode galvanic electrode or a band electrode for detecting a surface potential or current), an electromagnetic detector (e.g., an optical detector such as an infrared detector and visible light detector, as well as an x-ray detector, gamma-ray detector, etc.), a thermal detector, a pressure detector, an ultrasonic detector, a chemical detector, a magnetic detector, an x-ray detector, an accelerometer, a motion detector, etc. Other detectors in emerging sensor technology, such as laser Doppler, paper sensors, sensor tattoos, etc., can also be used.

Further, the wireless sensors in the network can include the same sensing components for redundancy or as required (e.g., in ECG applications where multiple electrodes at different body locations are required), or different sensing components for monitoring a plurality of different vital signs. For example, one sensor can include a pressure detector for monitoring the pulse rate, and another sensor can include an electrochemical detector for blood glucose level measurement (the glucose level can also be measured by an infrared detector or eye scanner). For another example, one sensor can include a surface-attached sensing component, such as an ECG electrode, and another sensor can include an implantable sensing component, such as an implanted intracardiac pressure transducer coupled to a heart chamber (e.g., the right ventricle). Thus, wireless sensors of different types for monitoring different vital signs can be conveniently worn by or implanted in the patient depending on the needs of care for the patient. These hybrid mix of sensors can also provide a caregiver with more comprehensive information regarding the patient's condition in an efficient manner.

Further, monitoring different vital signs simultaneously using a network of different types of wireless sensors can provide redundancy and improved robustness of monitoring quality as well as facilitate reconciliation of inconsistencies among the data gathered from different types of sensors (for different vital signs), reduce false alarm rates, etc. For example, certain vital signs can be considered as having higher priorities (e.g., because the sensors for monitoring these vital signs have higher reliability or accuracy), and as such, the data gathered for these vital signs can be given more weight when data gathered for other vital signs may suggest a different condition the patient is in. In addition, when implanted wireless sensors are used, especially those implanted relatively deep within the patient's body (e.g., in the patient's heart), one or more surface-attached sensors, e.g., those located near the implanted sensors, can be used to relay the signals acquired from the implanted sensors, e.g., to a master node, thereby providing potentially better quality signals for further processing and analysis. For example, for a wireless sensor implanted in a patient's heart chamber, another wireless sensor can be attached at the patient's chest to receive and re-broadcast the signals obtained by the implanted sensor. The wireless sensors can be further used in conjunction with certain medical devices worn by the patient (e.g., rehabilitating devices, robotics, prostheses, etc.), for collecting and transmitting sensed signals as a feedback or input for these devices so as to further enhance their functionalities.

Further, each wireless sensor includes a communication component configured for wireless communication with other sensors. For example, the wireless electrodes described in U.S. Pat. No. 7,979,111 (including the transmitting circuit, such as the remote telemeter 52), can be such a wireless sensor. A wireless sensor can include a mote as described in the above patent, or can include a fully integrated and functional communication circuit that includes an amplifier, a processor, a memory, a battery, and an RF module. Each or selected ones of the wireless sensors can further include a memory of suitable size, for example, 4 GB or 8 GB, to store a large volume or size of relevant medical records of a patient.

In some embodiments, the wireless sensors form a mesh network, where each sensor (also referred to as a “node,” “sensor node” or “regular node” hereinafter) not only captures and disseminates its own data, but also serve as a relay for other nodes, that is, the nodes in the mesh network collaborate with each other to propagate the data in the network. In certain embodiments, the mesh network further includes one or more control nodes (or master nodes), which communicate with any or all of the regular nodes. The master nodes can serve as a data acquisition, processing, and command center, and will be further described below.

The wireless sensor network can continuously monitor selected vital signs of the subject, and communicates the signals acquired from the sensing components via the communicating components of the sensors to a master node. Each of the wireless sensors can be programmed such that signals detected by the sensor falling into a predetermined (e.g., an acceptable or normal) range are not transmitted, or transmitted at a lower frequency. The acceptable range for signals for different patients and for each wireless sensor can be set individually, for example, based on the type of the sensor, the patient's condition, the therapy being used by the patient, etc. As described herein, the master node includes (A) a communication component configured to wireless receive signals from each of the plurality of wireless sensors, and send data and/or command to each of the plurality of wireless sensors; and (B) a monitoring unit coupled with the communication component. For example, the monitoring unit can include a readable medium and a processor coupled to the computer readable medium. The computer readable medium can store coded instructions for execution by the computer processor, which, upon the execution of instructions, carries out pre-designed tasks.

In some embodiments, the master node can be a PC or workstation computer equipped with a communication component, such as a dongle, for communicating with the wireless sensors. The master node can also include a portable device having a processor, a memory, a display and/or other audiovisual output capabilities to present information to a user, and capabilities of wirelessly communicating with the wireless sensors. In other examples, the master node can include a commercial portable computing device, such as a smart phone (e.g., an iPhone, an Android-based phone, a Windows Mobile-based phone, etc.), a tablet (such as an iPad, a Samsung Galaxy Tab, Google Nexus 7 or 10, etc.), or other similar devices. In further examples, the control and communication capabilities of a master node can also be implemented on one or more regular nodes to “upgrade” such regular nodes into “super nodes” that include both sensing capabilities and the functionalities of the master node as discussed herein. The monitoring unit of the master node can include a computer program designed to enable the monitoring unit to receive signals transmitted by the communication components of the wireless sensors, and to carry out appropriate functions based on such received signals, such as alerting the patient or a physician of an abnormal or emergency condition of the patient. However, it is to be understood that the monitoring unit can further integrate such received real time signals from the wireless sensors with the patient's past medical history and other relevant data (e.g., stored demographics, vital signs history, previous diagnosis, medications, allergies, etc.), and can provide capabilities such as making a suggestion for the diagnosis or treatment of the subject and validating a diagnosis or treatment proposed by a physician. Such medical history and other relevant data of a patient can be stored in a permanent storage medium (e.g., a hard drive, a solid state drive, a flash drive, or other types of memories) of the monitoring unit, or transmitted from a physician computer or a central server (such as a remotely located server operated by a healthcare provider, or a cloud server) accessible by the monitoring unit by wired and/or wireless communications. Also, the data acquired and stored by the master node can also be asynchronously or simultaneously uploaded to such a physician computer or remote server for long term storage and/or further analysis. In other words, the patient's EMR can be updated continuously or from time to time by incorporating the data gathered by the wireless sensor network. Without departing from the scope of the invention, this data can be stored either locally on the sensor or remotely anywhere on the network. As will be appreciated by one of ordinary skill in the art, depending on where the data is stored, storage capacity and throughput as well as other factors can be dramatically improved. The physician computer can further include a program that performs the tasks performed by a monitoring unit, such that the physician can directly monitor the patient's vital signs remotely, e.g., in the physician's office. Alternatively, the physician computer can be configured as a master node to directly communicate with the wireless sensors carried by the patient. Furthermore, the master node can transmit a patient's medical record stored or retrieved from a remote server or computer to selected wireless sensors having a storage medium having a sufficient storage capacity such that relevant patient data can be carried around on the wireless sensors worn by the patient and readily accessible in a clinical setting or another setting where the patient medical records are not otherwise available. Thus, one embodiment of the present invention provides an integrated system for acquiring, transmitting, analyzing, and utilizing vital signs (e.g., hemodynamic parameters, organ functions, blood test results) monitored in real time by wireless sensors worn by the patient together with the patient's medical records for clinical decision support and other patient health care objectives.

The system described in the present application can allow a physician to provide a correct diagnosis of symptoms of a patient. For example, although oxygen saturation for a healthy person is 90-100%, for a patient having a chronic obstructive pulmonary disease, the “normal” oxygen saturation is much lower. Thus, if a patient has an oxygen saturation level of lower than 90% (e.g., 86%) detected by the sensor network, the monitoring unit will not produce an alarm condition for low oxygen saturation, and can remind the physician if the physician makes a treatment recommendation under a mistaken belief regarding the “normal” oxygen saturation of this patient.

In another example, if a patient who has been taking beta-blockers has a low heart rate, e.g., lower than 40/min, as sensed by the wireless sensor and reported to a physician either remotely or in the physician's office, the system can make a diagnosis of the low heart rate condition and alert the physician that the patient should no longer be prescribed beta-blockers, but should consider other medicine or therapies.

As a further example, if a patient is taking an antibiotic, the appropriate dosage of the antibiotic can depend on the weight of the patient such that the patient's kidney function and liver function are not compromised. If the patient's weight has been mistaken when a prescription is given by the physician, the dosage can be incorrect as well, which can lead to ineffective treatment or undesired side effects. In this scenario, the system can validate the dosage prescribed by the physician based on a weight sensor worn by the patient, or by the patient's EMR information stored on a physician computer or downloaded from a server, and alert the physician if the dosage prescribed is not within a predetermined range appropriate for such a patient.

As a further example, if a patient visiting a healthcare provider complains about a fever and chest pains, the physician can check the patient's past medical records, which can be stored in the wireless sensors worn by the patient, including the patient's X-ray test result, and determine that the patient is suffering from a pneumonia. If the patient also has a history of alcohol (or other substance) dependence or abuse as indicated by the patient's past medical history, an antibiotic can be prescribed for the patient with an appropriate dosage based on this information as well as the patient's weight and other relevant information, or another therapy can be prescribed for the patient. The prescription can be entered into the monitoring unit of the system or a computer in the physician's office that is wirelessly coupled to the monitoring unit for sending and receiving information. Further, the system can further notify a pharmacy of the entered prescription, and direct the patient to fill the prescription at such a pharmacy. The physiological conditions of a patient of interest, including the effects of a prescribed therapy (including drug treatment, surgical procedures, etc.) on a patient can also be monitored by the patient or the physician by wireless sensors monitoring the vital signs relevant to the prescribed therapy, either in real time (e.g., the data acquired by the sensors can be transmitted in real time or intermittently to a monitoring device accessible by the physician (intermittent transmission refers to transmission of acquired data at a lower interval than the data acquisition or sampling rate by the sensor)), or at each visit to the physician's office by the patient.

In the following, wireless sensors including ECG electrodes suitable for acquiring electrophysiological signals related to cardiac function are used for illustrating the operating principles of the sensors and the network formed therefrom. In these sensors, each of the sensors include one or more electrodes which can acquire data related to the quality of ECG signal, such as the amplitude of a detected voltage, a detected current, and/or electrical skin resistance, and transmit such data to other sensors or the master nodes. The ECG electrodes can utilize off-the-shelf snap connector ECG electrodes to adhere to the thorax and to electrically connect to the skin.

In ECG applications, multiple wireless sensors are typically required, which are placed on the patient's body in predetermined locations. As will be further discussed below, these wireless sensors can further self-configure into a set or group which wirelessly send diagnostic quality ECG signals in a synchronous fashion to a master node, which can derive or synthesize ECG spectrum for display or other forms usable by a physician (or other users) based on the transmitted ECG signals. These sensors can also be configured to send and/or receive signals to/from the master node when a proximity criterion is satisfied, e.g., when the master node is within a predetermined distance from the wireless sensor, e.g., within 3 feet.

For illustration purposes and not limitation, the mesh or pseudo-mesh network formed by the sensors can be represented by a simplified block diagram as shown in FIG. 1. The illustrated network consists of six sensor nodes and a single master node 110. The sensor nodes can be divided into three clusters: cluster 120 (including node 1 and node 6), cluster 130 (node 2 and node 5), and cluster 140 (node 4 and node 9). The arrows in FIG. 1 represent communication paths between the nodes. As depicted in this example, the network supports at least two modes of communication:

Communication between the master node and each of the nodes, and

Communication between nodes.

Such a configuration allows for the sensor nodes make their own decisions and reconfigure the network independently of the master node.

The wireless communication within the mesh network can be based on proprietary communication stacks utilizing the principles of time domain multiple access (TDMA), with frequencies selected from various MICS bands (Medical Implant Communications Service frequencies) or from the ISM (Industrial, Scientific, and Medical frequency bands (900 MHz, 2.4 GHz, or 5.8 GHz)) as would be appreciated by one of ordinary skill in the art.

The components of the sample network shown in FIG. 1 are described in more detail below. It is understood that in addition to the example design as depicted and described herein, a single dedicated processor can control communications and data acquisition, as well as pre-processing of sensed signals with transmission of only extracted information. All of these functions may be performed in a dedicated application specific IC (ASIC) which performs data acquisition, analysis, communications protocol, and Radio-Frequency transmission and reception. Although specific communication channels and various networks are described it will be appreciated by one of ordinary skill in the art that the present invention should not be limited thereby and can also be implemented utilizing heretofore or hereafter developed without departing from the scope of the invention.

### A. Electrical Design for the Sensors

An exemplary block diagram of a regular node (or sensor) is shown in FIG. 2. In this example, two ECG electrodes 201 and 202 (e.g., a working electrode and a reference electrode) are used. The electrodes can be attached to the skin at predetermined locations and sense a surface voltage, thereby producing an ECG signal. The ECG signal is mixed with a square wave used to assess the quality of connection of the ECG electrodes to the skin. The mixed signal can be amplified in the instrumentation amplifier 220.

After amplification, the signal can be separated in low pass and high pass filters (230 and 232, respectively). The low bandwidth signal (ECG) can then be stripped of its 60Hz mains noise component by a 60Hz notch filter 240. Both low bandwidth signal (ECG) and high bandwidth signal (indication of the quality of connection of the ECG electrodes to the skin) can be fed to an A/D converter of the microcontroller 250 for digital processing. The processed signal is then sent to the RF module 280 for transmission to the master node and other nodes in the network. The RF module can be a highly integrated circuit which can run its own scripts and manage connection configuration, for example, it can send feedback and external commands to the microcontroller 250.

The circuit of the sensor can be powered by a Lithium Ion/Polymer battery 290. Supply voltage can be regulated by the switching regulator 296. The battery can be recharged by a battery charger 292 via a standard USB power supply. The battery for operating the electrodes can be rechargeable, which may be recharged using a standard Bluetooth® mains operated power supply connector, or remotely charged, e.g., by inductive charging. There can be a single battery for the electrodes and the circuit or they can each have their own power source. For safety reasons the battery temperature can be monitored by a thermal sensor 282 during charging.

In one example, the communication module of a sensor can include an RF module, e.g., SM200 (by Synapse®), which can be equipped with an on-board processor that allows for interpreting Python (high level programming) scripts defining operational modes. The scripts can be downloaded to the RF module over the air or through the module serial port. The module communicates in the 2.4 GHz ISM band. A serial port can be used for communication between the RF module and the processor. To allow for signaling events and reprogramming the unit using wires, a subset of module's available I/Os can be connected to the processor.

### B. Electrical Design for the Master Node(s)

The master node can include a RF module SM200 (U2, same type that used in the node), and USB [symbol] RS232 translator FT232BL (U3). FT2323BL provides an interface between RS232 and USB channels, and communicates with SM200 over RS232, and with a PC computer over USB. The circuit can be powered from a USB socket of the PC computer (which provides a 4.25V-5.25V power supply). RF module SM200 requires 3.3V power supply, and this voltage is generated by the LDO voltage regulator U1. To provide for reprogramming the RF module over RS232 and diagnostic mechanisms, several of the signals can be pulled to the test points, which allows for connecting diagnostic equipment. Optionally, an EEPROM memory U4 can be loaded onto the board to allow for more sophisticated configuration of the system (such as individual VID&PID identifiers for USB transmission). In this embodiment, the processor is considered as a part of the master node, however as would be appreciated by one of ordinary skill in the art, any general purpose computer or portable computing device can also be used without departing from the scope of the invention.

### C. Firmware Design

### 1. High Level Communication Protocol

As noted above, the wireless network can have a pseudo-mesh configuration. Any two nodes (including the master node) in the network can exchange information between each other, and any node in the network can broadcast information to all the remaining nodes in the network. Each node has its own unique identification number (NID). Firmware in a node (or master node) includes firmware for the PIC processor, and firmware for the RF module. The boot-up process can be controlled by the CPU, i.e., the CPU can control the RF module's reset line.

Some high level protocols for the system include the following:
1. Master node inquires each node about quality of their signals and builds an network image (i.e., set of active nodes). The image contains:
   a. Information about clusters, i.e., subsets of nodes which can substitute each other in case of a failure. (Information about clusters can be provided by the user based on the physical layout of the nodes on the patient's body.) Each cluster can be understood as a “virtual ECG electrode”. During normal operation, only one node in each cluster actively acquires data and sends the data to the master node. Clusters have their unique identification symbols (CID), for example, the clusters can be named A, B, C . . . .
   b. Information about hierarchy within clusters. Each node within cluster can be assigned a number defining its hierarchical position. The node of a lowest number can be the one which starts acquiring data. If it fails, the second highest number takes over, and so on.
2. Master node broadcasts the network image to the nodes.
3. Master node broadcasts time synchronization commands to the nodes.
4. Master node requests data from the nodes.
5. A node with the lowest number within the cluster of the lowest CID number broadcasts a block of data and signal quality report, then broadcasts a message indicating the completion of the broadcast. (Cluster CIDs comparison follows relation A<B<C . . . )
6. An active node in the cluster of a higher CID number takes over, sends a set of data and signal quality report to the master node and broadcasts a message indicating the completion of the broadcast.
7. The above steps 3-6 continue through the clusters.
8. After an active node in the cluster of the highest CID number sends data, the cycle starts again from (5).

The overall data acquisition speed can be kept at the level of data acquisition speed for active nodes (if a node does not have enough data to send, it stalls while waiting for a buffer to be filled). If a node within the cluster fails (for example, because of poor quality of contact of ECG electrodes to patient's skin, or battery voltage falling below a useful level), it broadcasts a message informing other nodes of the failure and activates a notification process (such as a blinking LED). The next node within a cluster can take over without involvement of the master node. The master node can be notified about the failure indirectly (by a change of the NIDs transmitting data), which allows for displaying additional information on a computer screen to notify medical personnel.

An exemplary algorithm useful for the system operates as follows:
One node within each cluster is assigned a role of the active node, i.e., a node transmitting data in a round robin cycle as discussed above (i.e., steps 3-6). This node is always a node with the lowest number within a cluster. The nodes make this decision independently (and concurrently) by analyzing the cluster layout stored in the node's memory.
If for any reason the active node is no longer capable of transmitting the data, the node sends a message to other nodes within its cluster. The transmission of this message is limited only to the member of the cluster, i.e., the nodes from other clusters and the master node are excluded from the list of the recipients of this message.
After receiving the message from the active node, each node compares its NID to the cluster layout. The node of the next lowest number takes over the role of the active node and starts sending data. This decision is made by each node individually, so the number of transmissions within a cluster can be minimized.

To avoid possible transmission collisions (which may take place if more than one node is transmitting at the same time), the timing of sending a message by the node no longer capable of transmitting the data is synchronized with the data transmission periods, i.e., the message is sent in the time slot allocated for the node for transmitting data.

At any time the system may be stopped and subsequently reconfigured by the master node.

Power consumed by a node depends on the state of the RF module of the node. The activity requiring the most power is data transmission. To minimize power consumption and maximize the operating time, the acquired data can be compressed before sending to the master node. For example, a sample received from the A/D converter can be 16 bits long. Since the A/D converter operates only on 12-bit data, the highest nibble of the sample is not used:

| Nibble 4 | Nibble 3 | Nibble 2 | Nibble 1 |
|---|---|---|---|
| 0000 | RRRR | RRRR | RRRR |

(RRRR means 4 bits of meaningful data.)

Since the highest nibble is always zero, a buffer containing data to send can be compressed using the following scheme:

Before compression:

| | | | | |
|---|---|---|---|---|
| WORD 1 | 0000 | A3 | A2 | A1 |
| WORD 2 | 0000 | B3 | B2 | B1 |
| WORD 3 | 0000 | C3 | C2 | C1 |
| WORD 4 | 0000 | D3 | D2 | D1 |

After compression:

| | | | | |
|---|---|---|---|---|
| WORD 1 | A3 | A2 | A1 | B3 |
| WORD 2 | B2 | B1 | C3 | C2 |
| WORD 3 | C1 | D3 | D2 | D1 |

This compression scheme guarantees 25% space saving and requires very little computing power.

### 2. Node RF Module Firmware

The master node can work as wireless RS232 bridge. The RF module in the master node can be configured to send RS232 data/commands from the PC computer over the air, and send data/commands received over the air to RS232 port of the PC computer. Such a configuration allows for easy interfacing with the PC computer.

Nodes can communicate in one of three modes: Primary mode. In this mode the RF module's switchboard is configured as (DS_TRANSPARENT, DS_STDIO), i.e., over the air stream is connected to SNAPPY STDIO stream. This mode allows for interpretation of commands and exchange of high level data relating to the communication configuration between nodes and the master node.

Secondary mode. In this mode the RF module's switchboard is configured as (DS_TRANSPARENT, DS_UART0), i.e., same way as in the master node (bridge mode). This mode is entered only for a limited time during sending ECG data from the processor to the master node. When the master node is ready to receive data, the node reconfigures switchboard to the bridge mode and sends a “ready” signal to the processor (by toggling the hardware (“h/w”) line). From this point the node cannot “see” wireless environment, and can only communicate with the processor by toggling h/w lines. After sending an entire block of data, the processor toggles h/w line, thus informing the RF module that the data transmission is completed. Then the RF module switches back to the primary mode.

Internal mode (CPU mode). In this mode the RF module's switchboard is configured as (DS_STDIO, DS_UART0). This mode is used during initialization when RF module exchanges information with the CPU over RS232 channel.

2a. Node Settings

Synapse RF200 RF modules can be individually configured to form a pseudo mesh network. Configuration parameters can be stored in the non-volatile memory of the node. The SN200 module can come with the configuration area preset to the default values. This section describes only non-volatile parameters of the node that need modification from their original values. The parameters need to be externally set for each of the nodes using network programming tools, such as Synapse Portal. References [ID x] in the titles define the relevant fields in the NVRAM storage area of the node. For illustration, an example test system having fewer than 10 nodes can have the settings described below (these values can be easily extended for larger systems).

Each node has a name [ID 8] set to “node_x”, where ‘x’ is an unique number [1 . . . 9]. Since there is only one master node in the network, it does not have to be equipped with unique name. Clusters can be created “on the fly” based on the multi-cast processed group defined by a 16-bit field in the configuration area of the node. Each bit represents a cluster. The following process describes the clustering in the network:
1. Master node broadcasts data, i.e., the data sent by the master node can be received by all the other nodes in the network. Transmission takes place over RS232 link.
2. Each node can communicate with to any subset of nodes in the network. Transmission between nodes is accomplished using SNAPPY STDIO stream. Transmission between a node and the master node is accomplished using RS232 stream.

The bits of multi-cast processed group word [ID 5] are set to the following values:
Bit 0—“broadcast all”, set to ‘1’ in all nodes (incl. master node).
Bit 1—“master node”, set to ‘1’ only in master node.
Bit x—“node x”, set to ‘1’ in the node of the corresponding number in the name.

The non-volatile memory of each network node can be set as follows.

Master node settings:
Multi-cast processed groups [ID 5]—0x0003 (dec. 3).
Multi-cast forwarded groups [ID 6]—0x0000 (dec. 0)
Device name [ID8]—since there is only one master node in the network, it is not used by the network and thus is irrelevant; by convention set to dongle x, where x is a single digit ASCII number.
Buffering timeout [ID 13]—0x0000 (dec. 0)
Buffering threshold [ID 14]—0x0078 (dec. 120)
Inter-character timeout [ID 15]—0x0000 (dec. 0)
Carrier Sense [ID 16]—True
Collision Detect [ID 17]—True
Collision Avoidance [ID18]—True

Node settings:
Multi-cast processed groups [ID 5]—a 16-bit value 0b00000aaaaaaaaa01, where bit ‘a’ is either 0 or 1. Only one bit ‘a’ is set to 1 (remaining ones are zeros). The set bit corresponds to the number of the node as in device name [ID 8]. The leftmost bit ‘a’ corresponds to ‘1’, the rightmost bit ‘a’ corresponds to ‘9’.
node_1 is 0b0000000000000101 (dec. 5),
node_2 is 0b0000000000001001 (dec. 9).
node_3 is 0b0000000000010001 (dec. 17),
node_4 is 0b0000000000100001 (dec. 33),
node_5 is 0b0000000001000001 (dec. 65),
node_6 is 0b0000000010000001 (dec. 129),
node_7 is 0b0000000100000001 (dec. 257),
node_8 is 0b0000001000000001 (dec. 513),
node_9 is 0b0000010000000001 (dec. 1025).
Multi-cast forwarded groups [ID 6]—0 (dec. 0)
Device name [ID8]—a string node_x, where x is a single digit ASCII number ‘1’ . . . ‘9’.
Buffering timeout [ID 13]—0x0000 (dec. 0)
Buffering threshold [ID 14]—0x0064 (dec. 100)
Inter-character timeout [ID 15]—0x0005 (dec. 5)
Carrier Sense [ID 16]—True
Collision Detect [ID 17]—True
Collision Avoidance [ID18]—True
   2b. Over the Air Commands

After powering up, a node performs initialization and a limited functionality test. Upon completion the node sends a string to the master node over the air. The string can have a following format: “node_a (b) Bc f/w v. d.dd/e.ee (group word f)”

where:
‘a’ is a number of the node (1 . . . 9).
‘b’ indicates result of the internal communication between the CPU and RF module: it is ‘+’ if the result was a success, ‘−’ if the result was a failure (in the latter case the node is not functional).
‘c’ indicates battery status: it is ‘+’ if the battery is charged, ‘−’ if the battery needs recharging (in the latter case data acquisition will not be possible).
‘d.dd’ is a RF module's firmware version.
‘c.cc’ is a CPU's firmware version.
‘f’ is a group word of the node (describing possible channels of communication of the node).

Primary mode commands (PMC) are sent between master node and any group or cluster of nodes in the network. The commands are sent over RS232 channel, thus are being exchanged between the PC application and a node. (The master node acts as a bridge passing data stream without intercepting/interpreting the stream.) The PMCs are ASCII based and always form a contiguous string of characters (without any spaces) ending with ‘\r’ or ‘\n’ characters (only one of such characters). The following table defines syntax of a PMC:

| | Length | | |
|---|---|---|---|
| | (char- | Allowed | |
| Field | acters) | Values | Description |
| Desti- | 1 | ‘0’ . . . ‘9’, | Defines the recipient of a command. |
| nation | | ‘a’ | The command may be addressed to |
| | | | master node (‘0’), any particular |
| | | | node (1 to 9), or to all nodes (‘a’). |
| | | | If a node finds during parsing that |
| | | | the destination is different than ‘a’ |
| | | | or NID of this particular node, the |
| | | | command is not processed. |
| Source | 1 | ‘0’ . . . ‘9’ | Defines the sender of a command. The |
| | | | command may be sent by master node |
| | | | (‘0’), or any particular node (1 to 9). |
| Opcode | 3 | Any pre- | Defines a command. |
| | | defined | |
| | | string of | |
| | | non-white | |
| | | ASCII | |
| | | characters | |
| Argu- | varies | Any pre- | The number of arguments, length and |
| ments | | defined | format of each arguments, and the |
| | | string of | meaning can be predefined and depend |
| | | ASCII | on particular command. Arguments are |
| | | characters | sent one by one forming contiguous |
| | | | string immediately following the |
| | | | opcode. |

An addressee may send a response to the sender, depending on the command. A response may vary in format, depending on the command, but always starts with a preamble. The table below defines format of a response.

| | Length | | |
|---|---|---|---|
| | (char- | Allowed | |
| Field | acters) | Values | Description |
| Desti- | 1 | ‘0’ . . . ‘9’, | Defines the recipient of a |
| nation | | ‘a’ | command. (See syntax of a PMC |
| | | | table for detailed description.) |
| Source | 1 | ‘0’ . . . ‘9’ | Defines the sender of a command. |
| | | | (See syntax of a PMC table for |
| | | | detailed description.) |
| Response | 3 | Always ‘res’ | Designates the message as a |
| flag | | | response. |
| Response | varies | Any predefined | Contains response. The length, |
| payload | | string of ASCII | format and meaning can be |
| | | characters | predefined and depend on |
| | | | particular command |

In case of an error, an addressee responds with an error message of the following syntax:

| | Length | | |
|---|---|---|---|
| | (char- | Allowed | |
| Field | acters) | Values | Description |
| Desti- | 1 | ‘0’ . . . ‘9’, | Defines the recipient of a command. |
| nation | | ‘a’ | (See syntax of a PMC table for |
| | | | detailed description.) |
| Source | 1 | ‘0’ . . . ‘9’ | Defines the sender of a command. |
| | | | (See syntax of a PMC table for |
| | | | detailed description.) |
| Error | 3 | Always ‘err’ | Designates the message as an error |
| flag | | | report. |
| Error | 2 | ‘00’ . . . ‘99’ | Defines error code. Code ‘00’ is |
| code | | | reserved and means ‘no error’. |

The commands can be called only if the node is not in the data acquisition mode.
A list of Primary Mode Commands are provided as follows. [D—destination ‘0’ . . . ‘9’, S—source ‘0’ . . . ‘9’, An—command argument (where ‘n’ is an index)]

Force 2-Node Network Configuration
Syntax: nd2
Arguments:
(none)
Description: allows for using network consisting of two clusters only (diagnostic).
Returns: (nothing)
Important: although the network consists of two clusters, three clusters have to be defined during network configuration. Cluster C is treated as ‘dummy’, and as such may be assembled of nonexistent nodes.

Start Single Node Data Transfer
Syntax: D, S, “dtr”
Arguments:
(none)
Description: Starts single node data acquisition mode. During this mode a node which receives this command starts acquiring data without synchronizing it with other nodes.
Returns: acquired data sent block after block. (Because the switching moment is not synchronized, first data block received by the computer may be corrupted.)

Start Multiple Node Data Transfer
Syntax: D, S, “daq”
Arguments:
(none)
Description: Starts multiple node data acquisition mode
Returns: acquired data sent block after block from all network clusters. The order of sending blocks may be random, for instance if the node stalls, there may be a gap in the incoming nodes followed by a series of multiple blocks sent from the same node.

Stop Data Acquisition
Syntax: D, S, “stp”
Arguments:
(none)
Description: Stops data acquisition.
Returns: if data acquisition stopped successfully returns D, S, ‘err10’, else returns D, S, ‘err11’.

Diagnostic Dump
Syntax: D, S, “dia”
Arguments:
(none)
Description: prints selected variables.
Returns: printout of variables:

| | |
|---|---|
| --- START DIAGNOSTIC --- | |
| Cluster A: 00000000 00000000 | <= bitmap of cluster A |
| Cluster B: 00000000 00000000 | <= bitmap of cluster B |
| Cluster C: 00000000 00000000 | <= bitmap of cluster C |
| Node 2 doesn't belong to any cluster | <= cluster to which the node belongs |
| Node is NOT an active node within its cluster | <= info whether the node is active within |
| cluster | |
| --- END DIAGNOSTIC --- | |

Introduce Yourself
Syntax: D, S, “int”
Arguments:
(none)
Description: sent to nodes by master node during network configuration
Returns: response D, S, ‘res’, followed by values of status (‘0’ or ‘1’), multi-case processed group ([ID 5]) and multi-cast forwarded group ([ID 6]).
Status describes result of node initialization. Value ‘1’ indicates that the node is functional, value ‘0’ indicates an error (a node which returns ‘0’ cannot be considered as a part of the network). Because returned values of [ID 5] and [ID 6] are formatted by SNAPpy Python, they may vary in length and sign being anything from −-32768 to 32767. To allow for unambiguous interpretation adjacent values are separated by commas. Example: 01res1,5,−1, means that node 1 responded to master node, it is functional, its [ID 5] contained value 0x0005, and its [ID 6] contained value 0xffff.

Clustering
Syntax: D, S, “clt”, N, Ai, . . . , Bj, . . . , Ck, . . .
Arguments:
N—number of Xy arguments following N
Ai, Bj, Ck—pairs consisting of a capital letter (for a test system: A, B or C) concatenated with a single digit (for the test system: 1 . . . 9). Each pair describes one node.
Description: The command (sent by master node) describes configuration of the network. The network described by the command consists of N nodes (for a test system N<10). The network is divided into clusters (groups of nodes), described by capital letters A, B, C . . . (for a test system number of clusters N<4, thus the clusters are: A, B and C).
Each pair represents a node in the network defining a cluster it belongs to. For example, a pair A1 defines node #1 as a member of cluster A. Note that in the system described herein clusters do not overlap (i.e., one node can belong to only one cluster).
Returns: response D, S, ‘res’, n
where: n is an ID of cluster to which the node belongs (A, B, C), or X if the node does not belong to any cluster described by the master node;
or D, S, ‘err20’ if network configuration is invalid (e.g., only two clusters are defined).

Synchronize Time
Syntax: D, S, “tmr”
Arguments:
(none)
Description: Timer synchronization. After receiving this command, the node zeros timer used for generating time stamp.
Returns: if node functional returns response D, S, ‘err00’, otherwise D, S, ‘err03’. (Used for verification by the master node that the node received command.)

Get Battery Status
Syntax: D, S, “bat”
Arguments:
(none)
Description: Command returns status of the battery (based on voltage threshold). At present the threshold is set to 3V.
Returns: if battery voltage is above threshold response D, S, ‘res0, otherwise D, S, ‘res0’.

Error Codes:
“err00”—no error
“err01”—invalid command
“err02”—invalid or missing argument
“err03”—command cannot be executed
“err04”—low battery
“err05”—command cannot be executed because of low battery voltage
“err06”—command cannot be executed because the node is not functional
“err10”—data acquisition has stopped (issued as a response to ‘stop data acquisition’ command, or if data acquisition was stopped as a result of the failure)
“err11”—data acquisition was already stopped (issued if there was an attempt to stop data acquisition, but the node has not been acquiring data)
“err11”—no more nodes in cluster (issued when the last functional node in cluster falls off)
“err20”—invalid network configuration
   2c. Node Tasks

| | Processor | |
|---|---|---|
| Task | Load | Comments |
| Send initial | One time | Upon request send to master node: |
| information to | | NID |
| master node | | Quality of connection information. |
| Receive | Depends on | Receive a string through RS232, |
| | command | parse it and execute it. Load |
| | | depends on the received command. |
| | | See “Over the Air Commands” |
| | | for possible cases. |
| Send ECG data | Small | Request data from the processor |
| | | and send it over the air through |
| | | RS232 |

2d. Over the Air Data

Data (ECG data) is sent from the node to master node in primary mode of communication. One transmission event consists of one block of data.

### 3. Node Processor Firmware

3a. Communication Between the CPU and the RF Module

Communication between CPU and RF module takes place either through dedicated hardware lines connecting modules (while in primary or secondary communication mode), or through RS232 channel (while in internal communication mode).

For communication through hardware lines connecting RF module and CPU, the lines connecting modules are shown in the table that follows:

| | | | RF | |
|---|---|---|---|---|
| | | CPU | Module | |
| Signal | Direction | Line | Line | Description |
| Data ready/ | CPU→RF | RB1 | PB6 (6) | Indicates data availability or transmission |
| transmission | | | | status. (See table below for details.) |
| status | | | | |
| Failure | CPU→RF | RB0 | PB5 (5) | Indicates error condition. If set, an error |
| | | | | took place. The nature of this error is |
| | | | | defined by ‘Data ready’ signal. (See the |
| | | | | table below for details.) |
| Data | RF→CPU | RB11 | PF2 (26) | Defines the meaning of ‘send buffer/ |
| acquisition | | | | synchronize time’ signal. (See the table |
| | | | | below for details.) |
| Send buffer/ | RF→CPU | RB12 | PF4 (28) | Depending on the state of ‘data acquisition’ |
| synchronize | | | | either data transmission handshake line, or |
| time | | | | strobe for timer synchronization. (See the |
| | | | | table below for details.) |
| Data ready | CPU→RF | RB7 | PF1 (25) | Indicates that data is available for the RF |
| | | | | module. |
| Send data | RF→CPU | RB2 | PB7 | The RF module sets this line high when it |
| buffer | | | | requests sending data buffer (rising edge). |
| | | | | Note: should be asserted only if Data Ready |
| | | | | line is high. |
| Sleep mode/ | CPU→RF | RF6 | PF0 (24) | Upon RF module reset: |
| Battery low | | | | if 1, the RF module enters deep |
| | | | | sleep mode. This is permanent state, and the |
| | | | | only way to wake the RF module up is by |
| | | | | applying reset. This option is used to |
| | | | | minimize the power consumption during |
| | | | | charging. |
| | | | | if 0, the RF module proceeds with |
| | | | | script execution. |
| | | | | While in code execution mode: |
| | | | | state 1 indicates that the battery |
| | | | | voltage falls below a predetermined |
| | | | | threshold and the battery should be |
| | | | | recharged. |
| | | | | state 0 indicates that the battery |
| | | | | voltage remains above the threshold and the |
| | | | | battery does not need recharging at this |
| | | | | moment. |

State description for ‘failure’ and ‘data ready’ lines:

| | Data rdy/ | | |
|---|---|---|---|
| Failure | Tran. status | Status | Description |
| 0 | 0 | Normal | No transmission in progress |
| 0 | 1 | operation | Transmission in progress |
| 1 | 0 | Abnormal | ECG signal quality error |
| 1 | 1 | operation | Buffer overflow error |

State description for ‘data acquisition’ and ‘send buffer/synchronize time’ lines:

| | Send buf./ | | |
|---|---|---|---|
| Data | sync | | |
| acq. | time | Status | Description |
| 0 | 0 | Data | Idle |
| 0 | 1 | acquisi- | Synchronize time. |
| | | tion | Immediately after detecting assertion of this |
| | | inactive | line (rising edge), CPU zeroes timer counter. |
| 1 | 0 | Data | Idle |
| 1 | 1 | acquisi- | Data transmission handshake lines. |
| | | tion | RF asserts ‘Send buffer’ line to signal CPU |
| | | active | to start sending data. After CPU detects that |
| | | | ‘Send buffer’ is high is asserts ‘Sending |
| | | | data’ line and starts transmitting buffer over |
| | | | RS232 line. When finished, CPU de-asserts |
| | | | ‘Sending data’ line. |
| | | | ‘Send buffer’ line is monitored by CPU only |
| | | | when there is no pending transmission. Once |
| | | | transmission is started, it continues until it’s |
| | | | finished. |
| | | | Typical protocol for sending one buffer is: |
| | | | RF module asserts ‘Send buffer’ line. |
| | | | CPU asserts ‘Sending data’ line and |
| | | | starts sending data. |
| | | | RF module de-asserts ‘Send buffer’ |
| | | | line. |
| | | | After the buffer is sent, CPU de-asserts |
| | | | ‘Sending data’ line. Since ‘Send buffer’ line |
| | | | is low, there is no new transmission started |
| | | | until it is asserted again. |

For communication through RS232 Channel (UART), RS232 signal sent from the RF module to CPU is used for:
Command ‘hey’ used for sending node number to CPU during initialization. The RF module sends a “pxhey\n” string, where ‘x’ is an ID of the node (‘p’ stands for ‘processor’). Having received the message, the CPU responds with the message “xprdy\n” if the CPU self-test has passed, of “xpflt\n” if the CPU self-test failed (‘x’ is the ID of the node).

The CPU retrieves and stores the node ID number to later include it in the data blocks sent to the master node.

The RF module attempts to send a “pxhey\n” string to the CPU several times. If the CPU does not respond, the RF node declares a failure.

3b. Synchronization and Timekeeping

All node processors are synchronized by an internal 32-bit timers clocked with the frequency of 62.5 kHz derived from the processor clock. The timer is reset to 0 by a command sent to the module by the master node. The reset command is broadcast which allows all the nodes synchronize within the network.

3c. Data Transfer

Data from the processor is transferred to the RF node in blocks of 200 digitized samples(instantaneous measurement of the value of a voltage) . . . . Each block has the following structure:

| | Length | | |
|---|---|---|---|
| Section | (bytes) | Value | Description |
| Preamble | 6 | D, S, “data” | Preamble allowing master |
| | | | node to accept data, and |
| | | | identify sender and kind |
| | | | of message. |
| Time stamp | 4 | 0 . . . | Number of “ticks” of the |
| | | 4294967295 | processor timer since last |
| | | | synchronization event in 16 μs |
| | | | increments. |
| Battery | 2 | varies | Raw 12-bit measurement of |
| voltage | | | battery voltage. |
| Payload | ¾ ∘ block | varies | Compressed block of data |
| | length | | |
| Checksum | 2 | varies | 16-bit CCITT CRC over all |
| | | | the blocks bytes with |
| | | | exception of the CRC. |

In the above table, Preamble is a 6 bytes long string of the following structure:
1. The first byte (ASCII character identified in the table above as D) indicates data destination. Its value corresponds to destination NID. In case of master node the value is 0.
2. The second byte (ASCII character identified in the table above as S) indicates source. Its value corresponds to a NID of a node sending data.
3. The third, fourth fifth and sixth bytes form a constant string “data” allowing for the recipient to determine the kind of the message.

During sending data, the node is put into a bridge mode (and thus cannot interpret/intercept data stream), the preamble must be built by the processor. To build a preamble the processor needs to know data destination and data source. Data destination is always 0, since master node is a recipient of ECG data. Data source is stored in the non-volatile memory of the RF module of the node. This value needs to be passed to the processor from the module over RS232 channel as a part of initialization of the node.

In the above table, Time stamp is a value of the timekeeping 32-bit timer in the processor. The timer is zeroed when the RF module receives synchronization signal from the master node and sets a h/w line signaling it to the processor. The timer increments every 16 μs, which allows for over 19 hours of continuous counting before is overwraps. Battery voltage is a raw 12-bit measurement taken with an A/D converter with 3.3V reference voltage. It means that the 12-bit scale (0x0000..0x0fff) reflects voltages 0V-3.3V. Voltages above 3.3V are clamped and read 0x0fff. The battery voltage can be calculated from the formula:

$$Vbat [ V ] = \frac{3.3 \cdot x}{4095}$$

where: x is an A/D reading. Payload is a compressed block of data. Compression can be very simple and based on the fact that in the 12-bit result of the ADC measurement the highest nibble is always zero. The length of the block expressed in words must be a multiple of 4. Checksum is a 16-bit CCITT CRC (x¹⁶+x¹²+x⁵+1 polynomial) calculated over preamble, time stamp and payload. All the multi-bytes variables are stored (are sent over the air) in little endian mode, i.e., a 32-bit value 0x12345678 will be sent as four bytes in the following order: 0x76, 0x56, 0x34, 0x12. The order of bits in particular bytes complies with the RS232 standard.
3d. Electrode Connection Contact Quality

To assess the quality of contact of the ECG electrodes to the skin, an AC signal of 10 kHz frequency is injected to the input of the ECG amplifier. The amplitude of the signal at the output of the amplifier is proportional to the resistance between the ECG electrodes indicating quality of their connection to the skin. The 10 kHz signal is filtered in hardware to strip it of the low frequency ECG waveform. Because of the finite bandwidth of the active filter/amplifier, the output signal is no longer a pure square wave, but because of the lack of high frequency components, the output signal has “rounded” edges. The changes in quality of connection of the ECG electrodes to the skin are usually gradual, so a few milliseconds delay in the process of their detection is acceptable. To assess the connection quality can be sufficient to measure minima and maxima of the signal and calculate the amplitude. If the amplitude is too big, the connection is poor.

The square wave frequency can be adjusted with 0.1% resolution. The data acquisition clock and the clock for the square wave come from the same source. With a square wave frequency set to a value slightly deviating from 10 kHz and sampling it with 200 Hz data acquisition frequency, the acquired signal will reflect the shape of the square wave because of aliasing. The deviation is chosen such that the frequency of the square wave signal is 10,012.5 Hz, which corresponds to approximately 25 sampling points per period.

For assessing the quality of contact of the ECG electrodes to the skin the minima and maxima of the signal can be recorded to calculate the peak-to-peak amplitude.

The algorithm for processing the signal is as follows:
(1). Wait for the rising slope (ΔV between two consecutive samples positive).
(2). Track sample values until a maximum is reached (i.e., until ΔV between two consecutive samples becomes negative). Store local maximum.
(3). Track sample values until a minimum is reached (i.e., until ΔV between two consecutive samples becomes positive). Store local minimum.
(4). Calculate the peak-to-peak amplitude for this period by subtracting local minimum from local maximum.
(5). Verify that the peak-to-peak amplitude remains in range. If not then:
Notify the RF module by asserting the line.
Halt data acquisition process.
If yes, AND data acquisition has been suspended, then:
   Notify the RF module by de-asserting the line; and
   Resume data acquisition process
(6). Go to (2).
   3e. Battery Charging

The battery is charged from the external 5V voltage applied to the USB connector either from the PC computer or the USB charger. When the 5V applied to the circuit, the battery charger U15 starts charging process and the line CHARGE_INDICATOR is driven low disabling the switching regulator U14. From that point the power supply switcher is disabled (i.e., the CPU and the RF module are not powered). When the battery is being charged, none of the two node's light indicators is active. When the charging process is finished, the line CHARGE_INDICATOR is released by the battery charger, the switcher starts operating, and the CPU and the RF module are powered again. When the 5V applied to the circuit, the state of the INPUT_VOLTAGE line changes, which can be detected by the CPU. When the CPU boots up, depending on the state of the line it can either boot up normally, or boots up into a low power mode. In this mode the RF module is in the deep sleep mode, and the CPU idles, blinking green LED every 3 seconds. When the battery voltage falls down to 4.1V, the battery charger activates again, and the charging cycle repeats.

3f. Buffers and Status Structures

Input buffers. A set of buffers each of which contains a time stamp and 200 samples. (Time stamp created when storing the first sample in the buffer.) Input buffers are filled by the ADC. Input buffers are unloaded by the processor for processing and storing in the output buffers. Buffer unloading takes place in predefined order.

Output buffers. A set of buffers each of which contains a block of data (Output buffers are filled by the processor with post-processed data from the input buffers. Output buffers are sent to the RF module by the DMA. Buffer sending takes place in the predefined order.

Input buffer status structure. Contains information indicating which buffer contains data ready for further processing, and error fields indicating buffer structure overfill (i.e., all the buffers were filled and there are no empty buffers left).

Output buffer status structure. Contains information indicating which buffer contains data ready sending to the RF module, and error fields indicating buffer structure overfill (i.e., all the buffers were filled and there are no empty buffers left).

Square wave parameters structure. Contains information about the most recent local minimum and maximum.

3g. Node Firmware Tasks

| | Processor | | |
|---|---|---|---|
| Task | Load | Resources | Description |
| Acquiring | Small | Timer 4, ADC | Free running timer 4 generates interrupts |
| ECG data and | | | on level 5 every 5 ms. Timer 4 ISR starts |
| input buffers | | | ADC conversion and returns. |
| management | | | ADC is configured such that automatically |
| | | | performs 16 conversions and generates in |
| | | | interrupt on level 6. |
| | | | ADC ISR reads ECG data, averages it, |
| | | | and stores in the input buffers. |
| | | | ADC ISR also manages the input buffer |
| | | | status structure by: |
| | | | Keeping track of filled elements in |
| | | | the buffer and switching to the next buffer |
| | | | when the current buffer is full, |
| | | | Creating a time stamp when the |
| | | | first sample of a new buffer is loaded, |
| | | | Signaling catastrophic situations |
| | | | such as buffer structure overfill. |
| | | | Immediately after averaging ECG data, |
| | | | ISR sets the ADC channel to the square |
| | | | wave input and triggers another |
| | | | conversion. (This conversion may be done |
| | | | in parallel with input buffer maintenance.) |
| | | | After the ADC conversion results are |
| | | | averaged, the result is processed and |
| | | | stored in the square wave parameters |
| | | | structure. |
| Sending data | Small | DMA3, | DMA3 is automatically sending data from |
| out and output | | UART1 | the output buffer to UART1 which is |
| buffers | | | connected to RF module. Hardware |
| management | | | control prevents RF module buffers |
| | | | overflow. |
| Data | Substantial | CPU | 1. Processing ECG input buffer data |
| processing | | | and transferring to the output buffers, in |
| | | | particular: |
| | | | Calculating CRC for the blocks. |
| | | | Maintaining buffers and sending |
| | | | alarm to the RF module in case of |
| | | | overflow. |
| | | | 2. Monitoring quality of connection |
| | | | to the skin and sending alarm to the RF |
| | | | module if needed. |
| | | | 3. Monitoring buffer overflow signal |
| | | | from RF module and suspending data |
| | | | transfer if needed. |
| | | | 4. Monitoring Synchronize Time line |
| | | | and resetting the real time counter. This |
| | | | task can be assigned an interrupt of the |
| | | | highest priority. |
| | | | 5. Receiving, parsing and executing |
| | | | commands received from RF module over |
| | | | RS232 link. |
| Generating 10 kHz | None | OC1, OC2 | OC1 is set to PWM mode synchronized to |
| complementary | | | itself. OC2 is set to the PWM mode |
| square waves | | | synchronized with OC1. |

### D. User Interface

The master node can be equipped with two control lights (preferably LEDs). The meaning of the lights is as follows. Green light indicates that the dongle is powered (i.e., it is plugged to the PC computer, and the computer is turned on). Yellow light indicates that data is transferred between the dongle and any of the nodes (in either direction).

The node can be equipped with two control lights (preferably LEDs). The meaning of the lights depends on the state of the node. If the node is turned off (i.e., it was put in the enclosure with a magnet turning the module off), both lights are off. If the node is being charged (i.e., the node is connected with the USB cable either with a PC computer or with a USB charger), the status of the light depends on the state of charging. If the battery is being charged, both lights are off. If charging process is finished, the green light blinks every 3 seconds.

When the battery is charged (charging process is finished), before the green light starts blinking, a power on sequence is displayed by both lights. Power on sequence is described in details below. If the node with a charged battery is left connected to the charger after the charging process is finished, the battery is no longer charged and after a certain time will discharge again. To prevent this, when the charge level drops below a predefined threshold, the node can automatically enter the charging phase again, i.e., the green light will stop blinking and both lights remain off until the spent charge is replenished.

Every time the node is turned on, the power on sequence is displayed by the lights. The sequence consists of a series of blinks: yellow-green-yellow-green-yellow-green.

If the node in turned on, the meaning of the lights is as follows: If the node is not acquiring data, both lights are off. If the node is acquiring data, the green light is blinking approx. every ½ second. If the node is inoperable (e.g., when ECG electrodes do not make good contact with the skin, or the battery is discharged below allowed level), the module blinks yellow light every 4 seconds.

The present invention is not to be limited in scope by the specific embodiments described herein. Various modifications of the invention in addition to those described herein will become apparent to those skilled in the art from the foregoing description and the accompanying figures.

One having ordinary skill in the art will recognize that the various mechanisms described for the preferred embodiments of the device may be adapted and interchanged between the preferred embodiments, without significantly impacting the structure and operation of the device. Use of the words “preferred embodiment” or “preferably” is not intended to imply that any other embodiment is less preferred or is not encompassed in the scope of the invention. Those skilled in the art will recognize that the present invention has many applications, may be implemented in many manners and, as such is not to be limited by the foregoing embodiments and examples.

Any number of the features of the different embodiments described herein may be combined into one single embodiment, the locations of particular elements can be altered and alternate embodiments having fewer than or more than all of the features herein described are possible. Functionality may also be, in whole or in part, distributed among multiple components, in manners now known or to become known.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention. While there had been shown and described fundamental features of the invention as applied to being exemplary embodiments thereof, it will be understood that omissions and substitutions and changes in the form and details of the disclosed invention may be made by those skilled in the art without departing from the spirit of the invention. Therefore, the appended claims are intended to cover conventionally known, future developed variations and modifications to the components described herein as would be understood by those skilled in the art.

What is claimed is:

1. A system for monitoring a subject, comprising:
a plurality of wireless sensors attachable to or implantable in the subject, each sensor comprising:
a sensing component configured to detect a signal corresponding to at least one hemodynamic condition of the subject;
a communication component configured to wirelessly transmit the detected signal to another of the plurality of wireless sensors;
a master node configured to receive signals from and transmit control commands to at least one of the plurality of wireless sensors.

2. The system of claim 1, wherein the plurality of wireless sensors are configured to form a mesh network.

3. The system of claim 1, wherein the vital signs include hemodynamic parameters selected from one or more of pulse oximetry, oxygen saturation, oxyhemoglobin saturation, blood glucose level, blood pressure, blood velocity, blood flow rate, respiratory rate, pulse rate, CO₂ level, drug concentration, blood protein concentration, heart rate, heart rhythm, heart rate variability, organic or inorganic substance concentration, cardiac activity, cardiac output, pH levels, pathogens and galvanic skin response.

4. The system of claim 1, wherein at least one of the plurality of wireless sensors includes a sensing component configured to detect a signal corresponding to a first hemodynamic parameter of the subject, and at least another of the plurality of wireless sensors includes a sensing component configured to detect a signal corresponding to a second hemodynamic parameter of the subject, the second hemodynamic parameter being different from the first hemodynamic parameter.

5. The system of claim 1, wherein at least one of the plurality of sensors is implantable.

6. The system of claim 1, wherein at least one of the plurality of sensors is configured to be attachable to the skin of a patient.

7. The system of claim 1, wherein at least a first sensor of the plurality of sensors is implantable, and wherein at least a second sensor of the plurality of sensors is configured to be attachable to the skin of a patient.

8. The system of claim 7, wherein the second sensor is configured to receive signals from the first sensor and relay the signals to the master node.

9. The system of claim 1, wherein the master node comprises a monitoring unit.

10. The system of claim 9, wherein the master node is a portable computing device.

11. The system of claim 1, wherein the sensing component includes at least one of an electromagnetic detector, a thermal detector, a pressure detector, an ultrasonic detector, an optical detector and a chemical detector, a magnetic detector, a laser detector, and an x-ray detector.

12. The system of claim 1, wherein the plurality of sensors are divided into two or more clusters, each cluster comprising two or more sensors, each of the two or more sensors configured to wirelessly communicate with the other of the two or more sensors and with the master node.

13. A method of monitoring a condition for a subject, comprising:
(a) detecting signals relevant to one or more vital signs of a subject by one or more wireless sensors attached to the skin or implanted in the body of the subject;
(b) wirelessly receiving, by a computing device, the signals from the one or more wireless sensors;
(c) making a diagnosis utilizing a processor of the computing device regarding a condition of the subject based on the signals received from the wireless sensors;
(d) accessing, by the computing device, at least one medical record of the subject; and
(e) determining, by the computing device, a health status of the subject based on the diagnosis made in step (c) and the medical record accessed in step (d).

14. The method of claim 13, wherein the signals are live signals acquired by the one or more wireless sensors in real time.

15. The method of claim 13, wherein the signals are for the vital signs relevant to one or more prescribed therapies of the subject reflected by the at least one medical record.

16. The method of claim 13, further comprising:
sending an alert to a physician according to the health status determined in step (e).

17. The method of claim 13, further comprising:
validating a prescription for the subject by a physician based on the health status determined in step (e).

18. The method of claim 13, wherein the medical record is stored on one of the plurality of wireless sensors.
